(19) United States
(12) Patent Application Publication
(10) Pub. No.: US 2019/0054216 A1
(43) Pub. Date: Feb. 21, 2019

(54) METHOD FOR LYMPHOLOGICAL LIPOSCULPTURE

(71) Applicant: MANUEL CORNELY, Dusseldorf (DE)

(72) Inventor: MANUEL CORNELY, Dusseldorf (DE)

(21) Appl. No.: 15/811,454

(22) Filed: Nov. 13, 2017

Related U.S. Application Data
(60) Provisional application No. 62/548,261, filed on Aug. 21, 2017.

(51) Int. Cl. A61M 1/00; A61B 5/00

(52) U.S. Cl. CPC A61M 1/006; A61B 5/418; A61B 8/0858; A61B 5/4872; A61M 2202/0405

(57) ABSTRACT

Procedures to achieve full subtotal, subcutaneous and suprafascial exeresis of fatty tissue between the skin and the musculature in lipedema and secondary lymphedema patients are provided. The procedures improve the patient's lymphological situation, such that no further manual lymphatic drainage or compression garments are required.

### RELATED APPLICATIONS

This application claims the benefit under 35 U.S.C. § 119(e) to U.S. Provisional Application No. 62/548,261, filed on Aug. 21, 2017, which is hereby incorporated herein by reference in its entirety.

### FIELD OF THE INVENTION

The present invention relates generally to liposculpture and more specifically to methods for treating lipedema and secondary lymphedema.

### BACKGROUND OF THE INVENTION

Lipedema is a disease that occurs almost exclusively in women. Lipedema results in the bilateral, symmetrical accumulation of fat. The accumulation of fat occurs primarily in the lower limbs from buttocks to ankles, but may also occur in the upper arms. Lipedema often leads to the legs becoming tender and being easily bruised.

Lymphedema is a disease in which lymph vessels are unable to adequately drain lymph fluid, usually from a leg or arm. Lymphedema can be either primary or secondary. Primary lymphedema occurs on its own, whereas secondary lymphedema is caused by another disease or condition. Secondary lymphedema is much more common than primary lymphedema and can occur in both women and men.

Historically, lipedema and secondary lymphedema have been misdiagnosed as simple obesity. Often, the aesthetic consequences of these diseases would be treated and the underlying causes of these diseases would be ignored. Thus, there is a need for treatments that alleviate and/or cure the painful consequences of these diseases.

### SUMMARY OF THE INVENTION

The present invention is based on the discovery of a method for achieving full subtotal, subcutaneous and suprafascial exeresis of fatty tissue between the skin and the musculature in lipedema and secondary lymphedema patients. This discovery improves the patient's lymphological situation, such that typically no further manual lymphatic drainage or compression garments are required in the patient's lifetime.

One embodiment of the present invention is to provide a method for performing lymphological liposculpture on a human body. The method includes performing at least a first, a second and a third operation. The first operation includes making incisions on an outer leg from the upper ankle joint to the inguinal ligament. The second operation includes making incisions on an arm from the wrist to the shoulder. A third operation includes making incisions on an inner leg from the upper ankle joint to the inguinal ligament.

In another embodiment, a wound produced on limb as a result of the incision is less than or equal to approximately 18% of the body's surface.

In another embodiment, a volume of tissue removed in an operation is less than or equal to eight liters.

In another embodiment, the incisions are less than 5 mm.

In another embodiment, the method includes administering a tumescent anesthesia locally to the limb in which the incision is being made.

In another embodiment, the method includes administering hyaluronidase with the tumescent anesthesia when the method is for treating secondary lymphedema.

In another embodiment, when the method is performed on a human body with lipedema, the volume of tumescent anesthesia administered in an operation is less than or equal to ten liters.

In another embodiment, when the method is performed on a human body with secondary lymphedema, the volume of tumescent anesthesia administered in an operation is less than or equal to five liters.

In another embodiment, an infiltration speed of the tumescent anesthesia is approximately 200 mL/minute.

In another embodiment, the tumescent anesthesia is administered into three vertical layers. The vertical layers include a lower layer, a middle layer, and an upper layer. The lower layer is the layer closest to the fascia.

In another embodiment, the tumescent anesthesia is allowed to take effect for approximately 1 to 2 hours before tissue is removed.

In another embodiment, the tissue in the lower layer is removed first. The tissue in the middle layer is removed next, and the tissue in the upper layer is removed last.

In another embodiment, a lymphatic drainage procedure is performed approximately four weeks after the method for performing lymphological liposculpture. The lymphatic drainage procedure includes massaging the body at or near the incisions.

In another embodiment, the massaging releases fluid from the incisions.

In another embodiment, the massaging is continued for approximately one hour.

In another embodiment, compression stockings are applied after the massaging is complete.

Other aspects and advantages of the invention will be apparent from the following description and the appended claims.

### DETAILED DESCRIPTION OF THE INVENTION

Before the present methods and compositions are described, it is to be understood that this invention is not limited to particular methods, compositions, and surgical conditions described, as such methods, compositions, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. The definitions set forth below are for understanding of the disclosure but shall in no way be considered to supplant the understanding of the terms held by those of ordinary skill in the art.

As used in this specification and the appended claims, the singular forms “a”, “an”, and “the” include plural references unless the context clearly dictates otherwise. Thus, for example, references to the “method for performing lymphological liposculpture” include one or more procedures/methods, and/or steps of the type described herein which will become apparent to those persons skilled in the art upon reading this disclosure and so forth.

As used herein, the terms “treating” or “treatment” or “alleviation” refers to therapeutic procedure/treatment, prophylactic and/or preventative measures, wherein the object is to prevent or slow down (lessen) the targeted pathologic condition or disorder. Those in need of treatment include those already with the disorder as well as those prone to have the disorder or those in whom the disorder is to be prevented. Treatment need not mean that the disease is totally cured.

As used herein, the term “patient” refers to a human.

The present invention relies on the following components as illustrative examples:

To inject the tumescent solution, a spray cannula (length 230 mm) with six outlet openings is used.

To remove the fat, a 300×4 mm “Double Mercedes” cannula, 10 mm hole, double-cut; and a 300×5 mm “Double Mercedes” cannula, 10 mm hole, double-cut are used.

The tumescent local anesthesia is prepared in 3-liter bags with NaCl 0.9%. The anesthesia contains 30 ml Xylocaine 2%, 30 ml Xylonest 2% with adrenaline 1:200,000, 20 ml sodium hydrogen carbonate, 2 ml adrenaline 1:1000 and 0.5 ml Volon A 40.

For the specific indication of treating secondary lymphedema on the leg or arm using this particular method, “Hylase-Dessau powder” is used in addition to the tumescent solution. Six thousand IU hyaluronidase to one liter of tumescent local anesthesia is used.

Lipedema patients affected on the arms and legs undergo surgery on all four limbs over the course of three operating sessions at intervals of no less than four weeks. The goal is to achieve full subtotal, subcutaneous and suprafascial exeresis of the fatty tissue between the skin and the musculature. The surgical areas are divided accordingly for this reason.

The first operation takes place on the outer legs i.e., the outer approximately 50% of the two legs. The operation is performed as far down as the upper ankle joint and as far up as the groin.

The second operation takes place on the arms; here, the operation is performed from the wrist to the axillary line and into the ventral shoulder area.

The third operation is performed on the inner legs; the limit is the upper ankle joint and the inguinal line.

These operations are organized predominantly around one key factor, which is that the wound produced on the respective limbs must not exceed approximately 18% of the body's surface. The operations are arranged in such a way as to ensure this. A second restricting factor is the amount of tissue removed must not exceed eight liters per operation, depending on the patient's anesthetic constitution.

The maximum dosage of tumescent local anesthesia in treating lipedemas should not exceed ten liters per session.

The maximum dosage for surgery on secondary lymphedemas on the legs or arms is five liters.

The procedure is carried out on the preoperatively marked surgical area while the patient is lying on the operating table. Analgosedation by the anesthetist ensures that the patient is pain-free. A stab incision is made using a no. 11 scalpel on the anterior medial line of the leg from the hip to the ankle at the uppermost puncture site i.e., in the groin area. A second stab incision is made in the middle of the thigh ventrally in the line. A third stab incision is made medially on the medial line of the lower leg. This applies equally to both sides. At this point, the tumescent local anesthesia is gradually pushed into the tissue using the spray cannula. An infiltration speed of approximately 200 ml per minute is crucial. The fluid must be infiltrated into the fatty tissue quickly. This form of local anesthesia is characterized by good diffusion into the tissue, which is a result of the pressure and the high speed. All the tissue to be anesthetized is filled until it becomes bulging and elastic. The inventor's methodological approach stipulates that the fatty tissue be divided into three vertical layers. In this case, first the lower vertical layer is filled with tumescence, then the needle is placed in the middle and finally in the topmost part of the fatty tissue. This ensures that the entire fat mass between the muscle fascia and the skin is completely saturated with tumescent local anesthesia. Typically, the expected effect from this amount of liquid is a marked swelling and a blanche effect of the entire tissue; it appears turgid and elastic. Once the marked surgical areas have been anesthetized in this way, the patient—after being awakened on the table—independently leaves the operating room on foot, accompanied by the anesthetist, and is placed on a couch in an adjoining room. The anesthetists are responsible for continuing the patient's anasthesiological care with regard to the patient's cardiovascular situation and breathing. The patient remains in this room for a maximum of approximately 1½ to 2 hours to wait for the tumescent local anesthesia to take effect. During this reaction period, the fatty tissue to be operated on is infiltrated further with the tumescent solution. The lipophilic substances dissolve the fat cell membranes as well as the fat cells themselves from their tissue compound, so that, after this exposure time, a liquefied amount of tissue is available for surgery.

Once back on the operating table, the patient undergoes surgery on the lipedema. This follows standard hygiene preparations under sterile conditions as well as analgosedation to ensure the patient remains pain-free. The first access point used is the upper puncture hole in the groin region: the 4 mm or 5 mm cannula is used to remove the tissue on the ventro-lateral side of the leg while the patient remains in the supine position. The strokes are performed with a vibrating cannula (vibration approximately 5,000 hertz). Suction is adjusted to approximately 600 mm Hg and the exudate flows into a vacuum container. The aspiration probe must be controlled properly to ensure that the liquefied tissue is removed gently and in sufficient quantity. In contrast to plastic surgery, the aim is to remove the tissue substantially and subtotally. For this reason, the lowest layer of the fatty tissue—i.e., the fatty tissue adjacent to the fascia, viewed horizontally—is removed first. Once this area has been completed, the surgeon proceeds with the probe into the medial-vertical fat layer and finally into the area directly below the skin i.e., the third fat layer, or the superficial layer. The full removal of fatty tissue from the limb is always performed in the same way: first profoundly, then medially and finally superficially. Suction is carried out using the cannula in such a way that it takes place just in the upper part, directly under the skin. Once again, this is unlike typical plastic surgery procedures, since in suction for cosmetic reasons, only the medial fat layer is removed—never the deep or superficial layer. This would not be sufficient in the case of a lipedema; in this case, all the layers must be removed. Leaving fat in one of the layers would compromise the result of the therapy.

In addition to an excellent knowledge of the fat layers, an excellent knowledge of the position of the lymph vessels as well as other vessels and nerve structures is crucial for this method to be effective. The surgeon must have an excellent three-dimensional picture of the extremities, otherwise the surgeon would not be able to operate on the lipedema properly. The cannula axis must always be aligned longitudinally, regardless of respective depth in the tissue. Stated differently, the cannula axis always runs parallel to the vessels or rather to the inaccessible bony structures lying deep below, so that under no circumstances can vessels be hurt, particularly the lymph vessels, by suction running crosswise to the axis.

At this point, the inventor emphasizes the importance of particular practice and caution exercised at a number of classic, critical anatomical places. In addition to the inguinal region and the well-known lacuna vasorum in the medial thigh-inguinal region, the lymphological clusters—referred to as “bottlenecks” in the literature—must be taken into account on the medial side of the knees. Only a parallel approach prevents injury to these lymph vessel structures. Once the lymphological liposculpture has been completed on the patient's first leg (the patient lying on his or her back) in the thigh region (starting centrally), the probe is guided to the next puncture site, that is, to the middle of the thigh between the knee and the groin. The leg is thereby slightly turned medially, so that the surgeon can perform suction with the cannula laterally at the side of the knee disc, up to the upper third of the lower leg. It is helpful to position the leg at 135°, in order to ensure unobstructed access and so that the cannula can be guided unhindered on a straight axis. This second step completely smooths the transition area from the thigh to the lower leg and restores the lateral contour of the knee and the knee disc. Finally, if applicable, the fatty tissue is removed directly over the knee disc towards the front of the shinbone. The surgeon now turns to the second leg, and, in the same way, starts to suction the fatty tissue of the thigh ventrally, but also laterally, as well as in the upper region of the thigh, towards the outer thigh. The procedure is the same: full removal of the fatty tissue using the suction probe in a strict longitudinal direction, first profoundly, then medially and subsequently superficially. The surgeon always uses his or her left hand (if right-handed) to feel for the correct position of the probe. Only by using the left hand can the trained surgeon identify whether the correct depth of the fatty tissue has been reached, and whether the correct amount of fatty tissue has been removed. After the upper portion of the second leg has been adequately suctioned, there is further suction at the second incision, in the same manner as the first leg. In this case, slightly rotating the leg inward is helpful for removing fatty tissue from the knee region up to the upper third part of the lower leg.

It should be emphasized here that the method can only be carried out if the surgeon is able to shift the patient's position on the table to meet the suction requirements. Unlike other conventional procedures, in the case of lymphological liposculpture, the patient must be positioned and turned in the operating area. This requires an experienced surgeon with an excellent grasp of three-dimensional anatomy.

At this point in the procedure, the patient is roused by the anesthetist. The patient is still pain-free, but somnolent. The patient is asked to turn to the right, so that the left leg is facing the surgeon laterally and from behind. To support this lateral position, the left leg is placed on an appropriate pad and the right leg beneath it is angled 90° at the knee, to ensure a nearly stable lateral position. Now commences the suction of the lateral and rear parts of the fatty tissue. The fatty tissue is removed in three layers as described above. The first access point is via the medial stab on the lateral mark on the lower leg. From here, first the back of the Bisgaard region (Achilles tendon loge) is completely emptied, and the junction between the foot and upper ankle is fully suctioned too. This produces what resembles a triangular configuration of sucked tissue. At a second access point (a new stab incision), suction is again performed on the calf, peripherally to center, from the upper ankle, so that all the transition points are smoothed up to the upper third of the lower leg. If necessary, the tube is once again inserted into the middle puncture hole on the thigh so that the exposed trochanter region can now be smoothed and suctioned. A transition point to the buttock region (gluteal region) is indicated here. This is carried out by suctioning different strokes in this region, so that the fatty tissue above the trochanter—i.e., the fatty tissue of the buttocks themselves—will match the leg. After completing this procedure, the patient is positioned on his or her left side, which is now complete. At this point, suction can be performed in a similar way on the outer side of the right leg—starting from the lower leg in a medial direction to the upper ankle including the Achilles tendon loge and its junction with the foot, then the calf (peripherally to center), and continuing to the trochanter region. The two puncture holes in the upper ankle area are also important for the subsequent drainage. There must be a good flow of fluids, consisting of tumescent solution but also secretion formed in the wound. The patient should now be completely awake on the operating table; the patient then walks into an adjoining room independently and then the bandaging takes place. The legs are bandaged peripherally to center; the puncture holes are covered with bandages and compresses. In this bandaged state, the patient leaves the operating rooms for his or her hospital bed.

The next day, when the patient returns, all the bandages are removed. After the surgeon examines the fresh surgical site, there is a final discussion and the patient is discharged.

Now begins the second part of the lymphological liposculpture procedure. This is carried out as accentuated manual lymphatic drainage with a local physiotherapist.

A prerequisite for the quality of this operation and its success is as follows: not only must the fat be substantially removed, but the lymphatic system must also be drained by means of lymphatic drainage and compression within the framework of lymphological liposculpture according to the inventor's protocol.

According to today's state of knowledge, the disease is based on a high volume transport failure in the lymphatic system. Patients suffer from increased lymph productivity in the fatty areas of the arms and legs and a disproportionately high transport capacity of the lymphatic vessels. However, this is not sufficient and the lymph becomes backed up between the fat cells. Removal of the fatty tissue, therefore, is only one tool on the way to levelling the imbalance between lymph production and lymph transport. Once all the operations have been performed, the patient describes no longer feeling any pressure pain on the arms and legs. The patient no longer requires further lymphatic drainage or compression if the procedure has been completed successfully. However, a four-week lymphological follow-up at home with a physiotherapist is absolutely necessary afterwards.

The inventor describes this procedure as accentuated manual lymphatic drainage, because the lymphatic drainage procedure typically entails a one-hour, full-body treatment. It starts at the classic spots in the neck region, preparing the terminus as well as the whole upper body. After carrying out this central decongestion, lymphatic drainage then proceeds to the arms and legs.

The emphasis of this lymphatic drainage, however, is on the postoperative drainage of the surgical areas, as a large amount of liquid continues to be produced for 3-10 days in the large wound area under the skin, which always corresponds to no more than approximately 18% of the body surface. The fluids must be drained off postoperatively through the open, as yet unsealed, incision openings in the skin. After ensuring that the workplace has been thoroughly prepared and that the skin has been cleaned properly (with the physiotherapist wearing gloves), the physiotherapist first drains the tissue at the surgical site. The physiotherapist does so in such a way as to remove liquid from the holes by gentle effleurage. After completing this first step—in which the emphasis is on flushing out the fluids—the physiotherapist performs the customary lymphatic drainage as described by Vodder. However, the physiotherapist does not place the same emphasis on the whole body, but rather drains the legs in particular, if the legs were operated on. This also applies similarly to the arms (described below); again, full-body drainage is performed, but this time focus is on the arms.

Finally, after this one-hour treatment, compression stockings are to be worn. In the first week, the patient must wear these compression stockings continuously for a 24-hour period, except when visiting the shower, toilet, undergoing manual lymphatic drainage or washing the compression garments. In the second, third and fourth week, the compression garment period is limited to approximately 12 hours, in which case the patient may choose to wear the compression garments either during the day or at night.

In the first week, lymphatic drainage is performed four times a week; in the second week, three times; twice in the third week and once in the last week, so that a total of 10 appointments are completed within four weeks.

This postoperative management is an effective part of the lymphological liposculpture according to the inventor's procedure and is a key factor in the lymphological success of the surgical procedure. In addition, the compression itself is essentially responsible for shaping the extremities without further lymphatic drainage.

It is important to emphasize once again that this procedure is not an aesthetic procedure; the goal is not to improve the appearance of the extremities. The goal is to improve the lymphological situation, meaning that patients will not need any more manual lymphatic drainage or compression garments for the rest of their lives. As lipedema patients, they are suffering from a disease. They are not patients whose focus is primarily geared towards changing the shape of their arms and legs for the sake of appearance.

Many patients recognize this distinction before undergoing the operation; after the operation, however, they tend to give priority to aesthetic matters. The surgeon who performs the lymphological liposculpture according to the inventor must refuse this.

Back to the surgical procedure: it is now four weeks since the first operation and the patient sees the surgeon again. After marking the fat regions on the arms, an appropriate tumescent local anesthesia is applied, followed by an initial circular suction. The incisions on the arms are made radially on the forearm in the wrist region, that is, in the elongation of the thumb, as well as laterally on the upper arm, above the elbow on the outside and inside. The tumescent local anesthesia is infiltrated into the fatty tissue between the muscle fascia and the skin via these incisions. Again, the tumescent solution must be pumped rapidly into all three vertically-pictured layers of the fatty tissue, starting right at the bottom on the fascia, then continuing into the middle and subsequently into the superficial part of the fatty tissue. The rule “profound/medial/superficial” generally applies in the lymphological liposculpture according to the inventor's method, as well as the rule of axial depth: starting profoundly, then medially and ultimately ending superficially in the liposuction procedure.

When operating on the arm, the anesthetist ensures the patient is pain-free—as with all three operations. The patient is given an analgosedation so that he/she can undergo the preparations and indeed the operation without any pain while asleep. After the tumescent local anesthesia has been applied, the same procedure takes place (the patient is placed in an adjoining room and remains there for a maximum of 1½ to 2 hours) until the tumescent local anesthesia takes effect as described above. The patient is then positioned on the operating table. As the patient sleeps, the arms are abducted at a 90° angle to ensure free access to both arms. The first surgical step is to remove the fatty tissue of the left forearm, using a 4 mm or 5 mm cannula, starting from the wrist to the elbow, until the lower third of the upper arm is reached. This ensures that no fatty tissue between the skin and the fascia is left. The second access then takes place in this position: on the arm above the elbow towards the shoulder.

The arm is then turned to the rear, so that the palm of the hand is now facing the surgeon. A stab incision of about 2 cm is made using a no. 11 scalpel in a central direction above the wrist i.e., in the region between the flow area of the arteries radialis and ulnaris between the skin and the fascia. From here, the fatty tissue is removed with the probe. The probes are adjusted as explained above (in the operation for fatty tissue on the leg). The speed of the vibration is set at approximately 5,000 hertz, and suction is performed at up to approximately 600 mmHg The probe is fed strictly parallel to the fascia; a mistake in the procedure at this point would directly affect the arm muscles and cause considerable damage. The second puncture is then made at elbow level, so that the region above the triceps and biceps in the upper arm can be operated on. Now, the arm is repositioned at a 90° angle so that the hand lies parallel to the patient's head and the volar palm of the hand is still pointing at the surgeon. Suction is then performed from the elbow towards the wrist, so that the lower transitional regions to the volar hand will be freed from fat.

The assistant now shifts position at the table and will stand on the other side of the patient facing the surgeon, so that the patient's arm, originally angled, is now guided over the patient's chest. The assistant holds the patient's hand so that the surgeon can perform suction dorsally from the incision at the elbow up to the wrist and make a final stab incision on the posterior axillary line in order to remove fat from the rear of the upper arm in the triceps and biceps area, with the arm now overstretched.

Once these procedures have been completed, the arm is put back into its relaxed position at a right angle to the patient's body on the table. The operator moves to the opposite arm and carries out the procedures in the exact same way until the fatty tissue has been completely removed on the second arm.

Finally, as discussed above, the arm is bandaged, paying particular attention to blood flow in the hands and mobility in one arm, so that the patient can move the hand (either as a right- or left-handed person) to the mouth. This is important for further care, particularly while in the hospital bed, so that the patient is able to eat and drink independently. The postoperative examination the following day is carried out in the manner described above. In this case, the subsequent four-week accentuated lymphatic drainage protocol is also obligatory, as it is a part of the lymphological liposculpture according to the inventor's method.

Over these four weeks, the patient wears compression stockings as described above: first for 24 hours, then for 12 hours during the day or at night, whichever the patient prefers. The patient undergoes 10 accentuated lymphatic drainage sessions.

After a further four weeks of lymphological postoperative care, the third and final procedure on the legs can be carried out (the inner legs). The anesthetized patient lies on the operating table, the tumescent local anesthesia is infiltrated via the stab incisions made using a no. 11 scalpel and previously anesthetized locally. For this purpose, the two stab incisions from the first operation are used on the thigh, and a further incision is made on the medial lower leg, in the middle area, between the upper ankle and knee. Local anesthetic is again inserted into the fatty tissue between the skin and the musculature, first profoundly, then medially, and then superficially. The tumescent solution causes the fatty tissue to become plump and elastic with liquid and displays blanching effects, which are partly due to the amount of the liquid and partly due to the adrenaline effect of tumescent local anesthesia.

Following exactly the same method, the tissue on the inner second leg is anesthetized. When the local anesthesia is applied, particular attention is paid to the dorsal parts of the two legs i.e., the rear parts up to the fold of the buttocks (gluteal fold) must be supplied with local anesthesia.

As discussed above, the patient is exposed to the local anesthetic. After approximately 1½ to 2 hours, suction is performed on these tumescented areas under analgosedation. First position: the leg is placed laterally on the table so that the surgical area is facing the surgeon. The surgeon is able to perform suction along the inner thigh with the probe, taking into account the special requirements of the vertical heights of the fatty tissue, as described above. Access via the second hole on the upper front of the thigh takes (in this position) the probe across the knee joint into the upper third of the lower leg in a parallel manner. The leg is positioned at an angle of approximately 135°, so that the probe passes parallel to the cluster of lymphatic vessels, which are arranged in parallel fashion in the medial knee region. A new incision is now made at the height of the medial knee joint gap in order to guide the probe first towards the upper ankle joint and thus ensure that parallel suction is performed on the lower ankle joint. From this position, it is also possible to suction superficially across the fatty tissue in the rear calf area, towards the Achilles tendon. The next incision is made on the medial side of the lower leg, so that, as described above, a triangular shape between the upper ankle joint and the medial lower leg can be suctioned. The fatty tissue will be completely removed. The surgeon then makes an incision on the medial side of the upper ankle joint and from there performs suction on the calf peripherally to center. In a final step, the surgeon removes the fatty tissue completely through the medial incision on the knee joint, also peripherally to center, in the inner thigh region up to the fold of the buttocks.

Similar procedures are applied to the opposite side of the second leg, so that by the end of the procedure, all the fatty tissue on the sides of the legs has been removed completely. Once the patient is awake, the patient is bandaged in the manner described above, admitted overnight and anesthesiologically monitored. The patient is seen postoperatively the following day by the surgeon and discharged into the hands of the physiotherapist for four weeks of accentuated lymphatic drainage.

In this way, the arms and legs of the lipedema patient are now completely free of fat. Postoperative examinations are carried out after approximately 6 and 18 months. Due to the size of the wound area caused by the operation, it is to be expected that wound secretions will leak from the existing punctures; this can last up to ten days. This is not lymph dripping (emphasis added); this is exclusively serous fluids such as liquidized hematoma, which occur naturally as a side effect of this method. The hematomas usually diminish within 10 to 14 days. The hematomas may extend into the fingers or toes; this is to be expected. Sore muscles can also be expected as another side effect. The patient receives perioperative intravenous antibiotic protection. Further medications are not necessary, other than a heparinisation with low-molecular-weight heparin for two days. In particular, no postoperative antibiosis is given.

The patient is encouraged to be mobile; staying in bed is not indicated and is counterproductive. But, in order to be safe, a sickness certificate is typically issued for one week in each case. However, depending on the patient's professional management, the patient can, of course, continue with work.

In sum, the quality of the procedure depends on the following:

1. A sufficient amount of tumescent local anesthesia being infiltrated into each surgical area.

2. The exposure time of the local anesthesia lasting at least one to two hours in order to transform the solid fat into a liquid, almost jellylike tissue.

3. Incisions not exceeding 4 or max. 5 mm in size and being made at different points.

4. The fatty tissue being completely removed in three layers, starting profoundly, then medially, and finally superficially.

In addition, an approximate four-week postoperative program must be followed comprising lymphatic drainage and compression.

A lipedema is a lymphological disease which must be treated with two components:

1. Removal of adipose tissue.

2. Training the lymphatic vessels to improve and restore normal lymph flow.

Based on these stipulations, a symptom-free rate of up to 95% has been obtained with the inventor's patients. These figures were determined in a large-scale study conducted in 2012 on approximately 600 patients over a 15-year follow-up period.

Conditions are different in the case of secondary lymphedema. The cause here is usually a stage II to III post-oncological edema at the limb in question i.e., a pitting (edema) to a non-pitting edema. The latter in particular is always accompanied by an increase in the size of the affected limb.

Although the lymphological liposculpture procedure is performed the same way on lipedema and secondary lymphedema, the conditions are somewhat different. When treating secondary lymphedema, an additional unit of up to approximately 6000 IU Hylase Dessau as hyaluronidase is added into the tumescent local anesthesia per liter of tumescent local anesthesia. This enzyme (Hylase Dessau/Hyaluronidase) dissolves and successfully softens the proteoglycan i.e., the solid substrate in secondary lymphedema.

The exposure time here is one to two hours. Positioning on the operating table, central anesthesia by the anesthetist in the form of analgosedation and access during the operation are also identical.

However, the postoperative treatment is different. In the case of secondary lymphedema, fourteen days of bandaging and lymphatic drainage is recommended. Lymphatic drainage is performed once a day. Patients are given postoperative antibiotic for ten days, which is different than when treating lipedema. In addition, the surgical procedures on the secondary lymphedema can be more extensive. This applies in particular when the hand and fingers or the foot or toes are characterized by the signs of secondary lymphedema. Tissue increase, including in the phalanges area, must be almost completely removed. This means that especially in the case of secondary lymphedema on the arm, as happens after breast cancer, the arm is positioned in such a way that the tissue is completely removed (after the appropriate tumescent local anesthesia and exposure time) by moving the 5 mm cannula from the puncture hole in the wrist region into the back of the hand and from there on to the fingers. A similar procedure applies to the alteration in the feet area, the back of the foot, and the toes in the case of corresponding secondary lymphedema. At present, the 5 mm cannula is the maximum size used for suction. By positioning the foot, it is possible to remove the tissue using the cannula over the joint into the outstretched foot up to the toes (stretched forward).

Finally, each finger or each toe must be bandaged individually. The bandaging is completed by applying foam pads to the back of the foot or the back of the hand, which ensure that the surfaces—now rid of subcutaneous tissue—do not fill with fluid again, but rather that eccentric compression predominates in this area.

After carrying out the postoperative phase consisting of 14 days of lymphatic drainage and compression, i.e., complex decongestive therapy, the need for complex decongestive therapy will gradually reduce on an individual basis. The interval between operations is always approximately four weeks.

In the case of secondary arm lymphedema, the operation result will be achieved by means of one operation, always using full circular suction.

In the case of lymphedema of the leg, it is necessary, due to the amount of tissue damage, to operate in two sessions, regardless of the amount of tissue that has to be suctioned i.e., first the outer legs (approximately 50%), then the inner legs (approximately 50%).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are relied upon and incorporated herein in their entirety.

1. Dr. Vodder's Manual Lymph Drainage: A Practical Guide 1st Edition, Jan. 6, 2011 (Thieme), by Dieter Wittlinger and Hildegard Wittlinger.

### 2. Cornely M E American Journal of Cosmetic Surgery 2014: Vol. 31, No. 2: 1-7.

3. Shiffman M A, Di Guiseppe A, eds. Liposuction Principles and Practice. Berlin, Germany: Springer Verlag; 2006: Chapters 1-10.
4. Shiffman M A, Di Guiseppe A, eds. Liposuction Principles and Practice. Berlin, Germany: Springer Verlag; 2006: Chapters 81-88.
5. Cornely M E, Schnabel H, Moser S, Kappelmeyer A, Marsch W Ch: Diagnostics and Differential Diagnostics of the Lipedema. Halle/Saale 1998.

### 6. Cornely M E: In Weissleder: Diseases of the Lymphatic Vessel System—Liposuction (Liposculpture), 1999.

### 7. Cornely M E: Slim Waist, Thick Legs, Thick Buttocks—No Diet Succeeds? CLEO 1999.

8. Cornely M E: Liposculpture. In: Weissleder H, Schuchhardt C: Diseases of the Lymphatic Vessel System. Cologne Viavital 2000; pp. 384-397.
9. Cornely M E: Liposuction with Lipedema. Journal for Lymphology 1/2001.
10. Cornely M E: Lipedema and the Differential Diagnoses Part 1-5. DERMAforum op-ed Column 2001.
11. Cornely M E: Diseases of the Lymphatic Vessel System. In: Weissleder H and Schuchhardt C, 2002.
12. Cornely M E: Liposuction with Lipedema—a Procedure with Highest Caution. Journal for Lymphology 1/2002.
13. Cornely M E: Lipedema and Lymphedema. In: Developments of Practical Dermatology and Venereology 2002, ed. by Plewig G, Prinz J, Berlin, Springer 2003; pp. 255-263.
14. Cornely M E: On the Terminology of Lipedema. In: MedReport Issue 46, published by Blackwell in November 2004. In: MAC 2004, Berlin 2004.
15. Cornely M E: Study “7 Years of Follow-up with Lipedema-Patients. Conservative versus Surgical Therapy.”
16. Cornely M E: Liposuction with Lipedema (Cellulite)—Follow-up with 140 Operated Patients after 7 Years. Abstract 27. Annual Conference of the VOD 2004.
17. Cornely M E: Liposuction with Lipedema (Cellulite)—Follow-up with 140 Operated Patients after 7 Years. Act Dermatol 2004.
18. Cornely M E: Liposuction with HIV-Patients. Abstract 27. Annual Conference of the VOD 2004.
19. Cornely M E: Cornely M E: Dermatology Practice Requires Lymphological Profile. The German Dermatologist 2004.

### 20. Cornely M E: Foundation of the “Working Group Surgical Lymphology”. MÄC 2004.

21. Cornely M E: Cellulites Treatment with Liposuction—Laser Removal of Tattoos—Facial-Rejuvenation. Aesthetic Tribune 2004.
22. Cornely M E: The Lipedema and its Treatment Options. Nuremberg 2004.
23. Cornely M E: Concept of Surgical Treatment of Lipohyperplasia Dolorosa, MedReport, published by Blackwell 2005.

### 24. Cornely M E: Thick Leg, Fat or Lipedema ? Duesseldorf 2005.

25. Cornely M E: Lipedema and Lymphatic Edema. In: Shiffman M, Cornely M E: Non-Cosmetic Applications of Liposuction. 2006, Chapter 3.
26. Cornely M E: Lymphology. JDDG 6/2006; pp. 564-579.
27. Cornely M E: Discussion around the Lipedema. Phlebologie 2005: 34: 271-2, p. 327.
28. Cornely M E: Liposuction of Lipedema. In: Shiffnian M, Cornely M E: Non-Cosmetic Applications of Liposuction. 2006, Chapter 86.
29. Cornely M E: Lymphological Liposculpture according to Prof. Cornely. German Patent and Trademark Office 2006 (Trademark Registration).
30. Cornely M E: Lymphological Liposculpture—What does Surgical Therapy offer in the Field of Lymphology, Hautarzt 2007 2007; 58: pp. 653-658.
31. Cornely M E: Series Dermaforum: Article 1: Lipedema, the often Misinterpreted Symptom Article 2: Surgical Lymphology enables the Healing of Lipedema Article 3: The Devastating Result of Oncological Healing: A Secondary Lymphedema. Article 4: Operat .Lymph.-Lymph. Liposculptur b.sekund. Lymphedema Dermaforum 2009.
32. Cornely M E: Fatter through Fat or Liquid—Lipohyperplasia Dolorosa vs. Lymphedema, Hautarzt 2010; 61: p. 873-879.
33. Cornely M E: Surgical Lymphology Part 1—The Lipedema; Part 2—About the Conservative and Surgical Therapy of Lipedema Named Lipohyperplasia Dolorosa; Part 3—The Preparation of the Paradigm Shift with the Secondary Lymphedema; Part 4: Lymphedema after Cancer Operation—Which Opportunities Does the Surgical Lymphology Offer? M. E. Cornely(1) Dermatology Surgery Dusseldorf Phlebologie 2011: 40 6: pp. 356-36.
34. Cornely M E: Lipedema 2012—15 Years after the Paradigm Shift.

### 35. Cornely M E: Puebla Book 2013; Chapter 1.

36. Cornely M E: Fatter through Lipids or Water: Lipohyperplasia Dolorosa versus Lymphedema; American Journal of Cosmetic Surgery; Vol. 31; No. 03-2014: pp. 189-195.
37. Cornely M E; 17 Years after the Paradigm Shift ; Dermaforum No. 5; May 2014.

### 38. CornelyM. E., Gensior M: Update Lipedema 2014, Cologne Lipedema Study, Lymphforsch 18 (2) 2014; pp. 66-71.

39. A Cornely M E: Lipedema 2014—an Update: Quality of Life Substantially improved; Dermaforum—No. 6—June 2014.

### 40. Cornely M E: Disorders of the Lymphatic System; Der Deutsche Dermatologe 2015; 63(2).

What is claimed is:

1. A method for performing lymphological liposculpture on a human body, comprising performing a first, second and third operation,
wherein the first operation comprises making incisions on an outer leg from about the upper ankle joint to the inguinal ligament,
wherein the second operation comprises making incisions on an arm from about the wrist to the shoulder, and
wherein the third operation comprises making incisions on an inner leg from about the upper ankle joint to the inguinal ligament.

2. The method of claim 1, wherein a wound produced on a limb as a result of the incision is less than or equal to approximately 18% of the body's surface.

3. The method of claim 1, wherein a volume of tissue removed in an operation is less than or equal to eight liters.

4. The method of claim 1, wherein the incisions are less than 5 mm.

5. The method of claim 1, further comprising administering a tumescent anesthesia locally to the limb in which the incision is being made.

6. The method of claim 5, further comprising administering hyaluronidase with the tumescent anesthesia when the method is for treating secondary lymphedema.

7. The method of claim 5, wherein when the surgical procure is performed on a human body with lipedema, the volume of tumescent anesthesia administered in an operation is less than or equal to ten liters.

8. The method of claim 5, wherein when the surgical procure is performed on a human body with secondary lymphedema, the volume of tumescent anesthesia administered in an operation is less than or equal to five liters.

9. The method of claim 5, wherein an infiltration speed of the tumescent anesthesia is approximately 200 mL/minute.

10. The method of claim 5, wherein the tumescent anesthesia is administered into three vertical layers, wherein the vertical layers comprise a lower layer, a middle layer, and an upper layer, and wherein the lower layer is the layer closest to the fascia.

11. The method of claim 10, wherein the tumescent anesthesia is allowed to take effect for approximately 1 to 2 hours before tissue is removed.

12. The method of claim 11, wherein the tissue in the lower layer is removed first, wherein the tissue in the middle layer is removed next, and wherein the tissue in the upper layer is removed last.

13. A lymphatic drainage procedure to be performed approximately four weeks after the method of claim 1, said lymphatic drainage procedure comprising massaging the body at or near the incisions.

14. The lymphatic drainage procedure of claim 13, wherein the massaging releases fluid from the incisions.

15. The lymphatic drainage procedure of claim 13, wherein the massaging is continued for approximately one hour.

16. The lymphatic drainage procedure of claim 15, wherein compression stockings are applied after the massaging is complete.
